SEQUENCE LISTING (32 sequences)

SEQ ID NO: 1 - DNA, 20 bp, Artificial sequence
  PGC-1a forward primer
gtccttcctccatgcctgac

SEQ ID NO: 2 - DNA, 20 bp, Artificial sequence
  PGC-1a reverse primer
gactgcggttgtgtatggga

SEQ ID NO: 3 - DNA, 20 bp, Artificial sequence
  ERRa forward primer
gaggtggaccctttgccttt

SEQ ID NO: 4 - DNA, 20 bp, Artificial sequence
  ERRa reverse primer
ggctaacaccctatgctggg

SEQ ID NO: 5 - DNA, 20 bp, Artificial sequence
  NRF-1 forward primer
cttcatggaggagcacggag

SEQ ID NO: 6 - DNA, 20 bp, Artificial sequence
  NRF-1 reverse primer
atgaggccgtttccgtttct

SEQ ID NO: 7 - DNA, 20 bp, Artificial sequence
  Tfam forward primer
gagcgtgctaaaagcactgg

SEQ ID NO: 8 - DNA, 20 bp, Artificial sequence
  Tfam reverse primer
ccacagggctgcaattttcc

SEQ ID NO: 9 - DNA, 20 bp, Artificial sequence
  MyoD forward primer
ccgtgtttcgactcaccaga

SEQ ID NO: 10 - DNA, 20 bp, Artificial sequence
  MyoD reverse primer
gtagtaggcggtgtcgtagc

SEQ ID NO: 11 - DNA, 20 bp, Artificial sequence
  Myogenin forward primer
gaggaagtctgtgtcggtgg

SEQ ID NO: 12 - DNA, 20 bp, Artificial sequence
  Myogenin reverse primer
ccacgatggacgtaagggag

SEQ ID NO: 13 - DNA, 20 bp, Artificial sequence
  SDHb forward primer
actggtggaacggagacaag

SEQ ID NO: 14 - DNA, 20 bp, Artificial sequence
  SDHb reverse primer
gttaagccaatgctcgcttc

SEQ ID NO: 15 - DNA, 21 bp, Artificial sequence
  CytC forward primer
gggcctcgttagtgcagcagg

SEQ ID NO: 16 - DNA, 22 bp, Artificial sequence
  CytC reverse primer
gggctcccagaaaaggttgcct

SEQ ID NO: 17 - DNA, 20 bp, Artificial sequence
  COX2 forward primer
acgaaatcaacaaccccgta

SEQ ID NO: 18 - DNA, 20 bp, Artificial sequence
  COX2 reverse primer
ggcacaacgactcggttatc

SEQ ID NO: 19 - DNA, 20 bp, Artificial sequence
  COX4 forward primer
actaccccttgcctgatgtg

SEQ ID NO: 20 - DNA, 20 bp, Artificial sequence
  COX4 reverse primer
gcccacaactgtcttccatt

SEQ ID NO: 21 - DNA, 20 bp, Artificial sequence
  ATP5g1 forward primer
tggggaccagggcagccatt

SEQ ID NO: 22 - DNA, 20 bp, Artificial sequence
  ATP5g1 reverse primer
agggcttgctgcccacacat

SEQ ID NO: 23 - DNA, 20 bp, Artificial sequence
  PPAra forward primer
atccagggttcagtccagtg

SEQ ID NO: 24 - DNA, 20 bp, Artificial sequence
  PPAra reverse primer
gcttagggacagtgacaggt

SEQ ID NO: 25 - DNA, 20 bp, Artificial sequence
  PPARd forward primer
gttcctctgaccctgtcctc

SEQ ID NO: 26 - DNA, 20 bp, Artificial sequence
  PPARd reverse primer
ccagcaagtttcaagccact

SEQ ID NO: 27 - DNA, 20 bp, Artificial sequence
  UCP1 forward primer
cggaaacaagatctcagccg

SEQ ID NO: 28 - DNA, 20 bp, Artificial sequence
  UCP1 reverse primer
ctgaccttcacgacctctgt

SEQ ID NO: 29 - DNA, 20 bp, Artificial sequence
  SIRT1 forward primer
tgccatcatgaagccagaga

SEQ ID NO: 30 - DNA, 20 bp, Artificial sequence
  SIRT1 reverse primer
aacatcgcagtctccaagga

SEQ ID NO: 31 - DNA, 20 bp, Artificial sequence
  GAPDH forward primer
aactttggcattgtggaagg

SEQ ID NO: 32 - DNA, 20 bp, Artificial sequence
  GAPDH reverse primer
ggatgcagggatgatgttct

(19) United States
(12) Patent Application Publication
(10) Pub. No.: US 2021/0161858 A1
(43) Pub. Date: Jun. 3, 2021

(54) COMPOSITION FOR PREVENTING AND TREATING MUSCLE DISEASE, IMPROVING MUSCLE FUNCTION OR ENHANCING MOTOR PERFORMANCE COMPRISING HYDRANGENOL OR HYDRANGEA EXTRACT AS ACTIVE INGREDIENT

(71) Applicant: COSMAXBIO CO., LTD., Jecheon-si (KR)

(72) Inventors: Sun Hee LEE, Seongnam-si (KR); Yu Kyong SHIN, Yongin-si (KR); Hyoun Jea KIM, Yongin-si (KR); Hye Shin AHN, Bucheon-si (KR)

(21) Appl. No.: 17/258,540

(22) PCT Filed: Nov. 27, 2018
(86) PCT No.: PCT/KR2018/014704

§ 371 Date: Jan. 7, 2021

(30) Foreign Application Priority Data
Jul. 11, 2018 (KR) 10-2018-0080798

(51) Int. Cl. A61K 31/366; A61K 36/185; A23L 33/105; A23L 29/00

(52) U.S. Cl. CPC A61K 31/366; A23L 29/035; A23L 33/105; A61K 36/185

(57) ABSTRACT

Provided is a composition for preventing and treating muscle diseases, improving muscle functions, or enhancing motor performance that comprises hydrangenol or a hydrangenol-containing extract of Hydrangea as an active ingredient. The hydrangenol or Hydrangea extract containing hydrangenol is derived from natural materials and thus can be used safely without any adverse effect, and the composition comprising the hydrangenol or Hydrangea extract can be used beneficially as a medical, food, quasi-drug, or cosmetic composition having a good effect of preventing and treating muscle diseases, improving muscle functions, and enhancing motor performance.

### TECHNICAL FIELD

The present invention relates to a composition for improving muscle functions and enhancing motor performance, and more particularly to a pharmaceutical composition for preventing, improving or treating muscle diseases, a health functional food for preventing and improving muscle diseases, and a cosmetic composition for improving muscle functions that include hydrangenol or a Hydrangea extract.

### BACKGROUND ART

Muscles are a tissue that arises from the stem cells of the mesoderm. They are divided into three types: skeletal muscle, cardiac muscle, and smooth muscle, which produce force and motion in their respective positions. Among them, the skeletal muscle accounts for about 40% of the total body weight and consists of muscle cells, myoblasts. If not used consistently, these muscles are deteriorated in their functions due to aging and reduced to cause fatigue (J. Appl. Physiol. 2003, v. 95, pp. 1717-1727).

Muscular strength is defined as the maximum amount of force a muscle can produce against some form of resistance (e.g., weight, force) in a single effort, that is, the maximum force that a muscle or muscle tissue can exert at a single effort. Motor performance refers to the ability to perform a motor task using the muscular strength. The muscular strength may decline due to aging, fatigue, lack of exercise, various diseases, increased stress, nutritional imbalance, alcohol, smoking, etc. The decline of muscular strength can deteriorate the motor performance and lead to decreased physical strength, obesity, hyperlipidemia, high blood pressure, etc.

Exercise increases the biosynthesis of mitochondria, organelles that produce energy, and regulates the size and number of muscle fibers and the synthesis of muscle proteins to increase the muscle mass. The synthesis of mitochondria and the muscle mass are major factors that determine the functions of the muscle and improve the motor performance.

Hydrangeas are a genus of broad-leaved dwarf cultivars in the Saxifrage family that grow wildly in Korea, where they are known as Hydrangea serrata or Hydrangea macrophylla. Besides, their leaf is an edible part as found in the list of food materials according to the Ministry of Food and Drug Safety in South Korea. The leaf extracts of Hydrangea are usable as specified under the names of the Chinese Cosmetic Ingredients and the INCI (International Nomenclature Cosmetic Ingredients). The leaf is called “Mountain Hydrangea leaf” as an herb of the oriental medicine and has long been used for treatment of chronic bronchitis, relief of cough and phlegm, anti-inflammation, detoxification, etc.

Hydrangenol is a component mostly found in Hydrangeas (JP-0029934); molecular weight: 256.25 g/mol, IUPAC name: 8-hydroxy-3-(4-hydroxyphenyl)-3,4-dihydroisochromen-1-one. Further, its derivatives are (−)-hydrangenol 4′-O-glucoside and (+)-hydrangenol 4′-O-glucoside. In addition, Hydrangenol is reported to have functions of skin whitening (JP-0007546) and anti-inflammation (Kim, H. J, et al., “Hydrangenol inhibits lipopolysaccharide-induced nitric oxide production in BV2 microglial cells by suppressing the NF-_{K} B pathway and activating the Nrf2-mediated HO-1 pathway”, International Immunopharmacology Vol. 35, pp. 61-69, 2016, 1567-5679).

Yet, there have never been made studies on the direct mechanisms of the actions of the extracts and the component to prevent muscle diseases, improve muscle functions, and enhance motor performance. For this reason, the inventors of the present invention have performed research on the direct efficacy of the component (hydrangenol) in a composition for preventing muscle diseases, improving muscle functions, and enhancing motor performance.

As a result of the studies made in an attempt to solve the problems with the prior art, it is suggested that a composition containing hydrangenol and a Hydrangea extract as active ingredients increases expression and activity of MyoD and Myogenin as transcription factors involved in differentiation and development of muscle to protect muscle cells and improve muscle functions, increases expression and activity of mTOR and p70S6K to gain the muscle mass, activates AMPK, PGC-1α, and SIRT1 to increase mitochondrial biogenesis, and furthermore, promotes the activities of mitochondrial electron transport chain (ETC) and mitochondrial synthases, COX and ATG5g1, thereby having an effect of preventing muscle diseases, improving muscle functions and enhancing motor performance, thus completing the present invention.

### CITED DOCUMENTS

### Patent Documents

Patent Document 1: KR1020170124426 A

Patent Document 2: JP-0007546 A

### Non-Patent Documents

Non-Patent Document 1: Kim, et al., “Hydrangenol inhibits lipopolysaccharide-induced nitric oxide production in BV2 microglial cells by suppressing the NF-_{K} B pathway and activating the Nrf2-mediated HO-1 pathway”, International Immunopharmacology, 2016, 35: 61-69

### DISCLOSURE OF INVENTION

### Technical Problem

It is therefore an object of the present invention to provide a composition for preventing and treating muscle diseases, improving muscle functions, or enhancing motor performance that comprises hydrangenol or a Hydrangea extract as an active ingredient.

### Technical Solution

To achieve the above objects, the present invention provides a composition for preventing and treating a muscle disease, improving a muscle function, or enhancing a motor performance that comprises hydrangenol of the following Chemical Formula 1 or a Hydrangea extract containing the hydrangenol as an active ingredient:

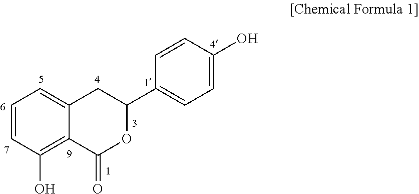 [SMILES: O=C1OC(c2ccc(O)cc2)Cc2cccc(O)c21]

In the present invention, the hydrangenol may be isolated and purified from the natural materials of Hydrangea serrata or Hydrangea macrophylla of Hydrangeas. The Hydrangeas may be at least one selected from the group consisting of the whole, woody root, stem, branch, leaf, seed, and fruit of Hydrangeas. Preferably, the Hydrangeas may be the leaf of Hydrangeas.

In the present invention, the Hydrangea extract may be obtained by any conventional extraction method for natural plant extraction, including hot water extraction, solvent extraction, distillation extraction, or supercritical extraction. Preferably, the Hydrangea extract is obtained by extraction with water, an organic solvent, or a combination of both. The organic solvent may be at least one selected from the group consisting of alcohols having 1 to 4 carbon atoms, such as ethanol, methanol, isopropanol, and butanol; preferably ethanol; and more preferably hot water (Refer to Example 1).

In the present invention, the term “muscle” as used herein refers to a tissue composed of an aggregate of muscle cells. If not limited to a specific type of muscle, the muscle is preferably a skeletal muscle, which inclusively refers to tendons, muscles, and sinews.

In the present invention, the term “improving a muscle function” as used herein means that the functions of the muscle are improved, preferably by expressing the factors involved in the production of muscle at a protein or mRNA level and inducing the protein synthesis and the formation of muscle fibers to increase the skeletal muscle mass.

In the present invention, the term “motor performance” as used herein refers to the ability to perform a motor task using muscles. The term “enhancing a motor performance” as used herein may mean at least one selected from the group consisting of increasing muscle strength, enhancing endurance, promoting energy metabolism, and improving cardiopulmonary functions. Preferably, enhancing a motor performance may include all of increasing muscle strength, enhancing endurance, promoting energy metabolism, and improving cardiopulmonary functions.

In the present invention, the term “muscle disease” as used herein refers to a disease reported in the related art as a muscle disease caused by deteriorated muscle function, muscle loss, or muscle degeneration. Preferably, the muscle disease is at least one disease selected from the group consisting of muscular atrophy, muscular dystrophy, myasthenia, muscle degeneration, and sarcopenia.

In the present invention, there is provided a composition for preventing and treating a muscle disease, improving a muscle function, or enhancing a motor performance that comprises the hydrangenol or the Hydrangea extract in an amount of 0.0001 to 100 wt. % with respect to the total weight of the composition.

In the present invention, the composition is a preparation for oral application and has at least one dosage form selected from the group consisting of tablet, granule, pill, capsule, liquid, jelly, or gum.

In the present invention, the composition is a preparation for cutaneous application and has at least dosage form selected from the group consisting of toner, essence, nourishing cream, moisturizing cream, spot, gel, lotion, ointment, patch, and aerosol.

In the present invention, the composition may be a composition for various uses, including a pharmaceutical composition, a health functional food composition, a quasi-drug composition, or a cosmetic composition.

The cosmetic composition of the present invention may further include at least one cosmetically acceptable carrier that is blended into a general skin cosmetic composition. It may be also appropriately blended with typical components, including, but not limited to, oils, water, surfactants, moisturizers, lower alcohols, thickeners, chelating agents, colorants, preservatives, and fragrances.

The pharmaceutical composition or the health functional food composition according to the present invention may further include appropriately selected carriers, excipients, or diluents generally used in the manufacture of pharmaceutical compositions. The pharmaceutically acceptable carriers, excipients or diluents may include, but not limited to, at least one selected from the group consisting of lactose, dextrose, sucrose, calcium silicate, cellulose, methylcellulose, microcrystalline cellulose, polyvinylpyrrolidone, water, methylhydroxybenzoate, propylhydroxybenzoate, talc, magnesium stearate, and mineral oil.

The composition of the present invention may be administered orally or parenterally. For parenteral administration, it may be formulated into a dosage form of cutaneous preparations; intravenous, intramuscular, or subcutaneous injections; transdermal preparations; or nasal inhalants, according to the methods known in the related art.

The pharmaceutically effective amount and effective dosage of the pharmaceutical composition according to the present invention may vary depending on the formulation method, the mode of administration, the administration time and/or the route of administration of the pharmaceutical composition. They may also vary by different factors, including the type and intensity of the reaction intended by the administration of the pharmaceutical composition, the type, age, body weight, general health condition, the symptom or severity of disease, gender, diet, and excretion of the subject to which the composition is administered, the ingredients of another drug composition administered to the same subject along with the pharmaceutical composition in a simultaneous or asynchronous manner, and similar factors well known in the medical field. Those of ordinary skill in the art may easily determine and prescribe the effective dosage for the intended treatment. The pharmaceutical composition of the present invention may be administered once or multiple times daily. Therefore, the above dosage does not limit the scope of the present invention in any aspect. Preferably, the dosage for the hydrangenol of the pharmaceutical composition according to the present invention is 0.01 to 20,000 μg/kg/day, more specifically 1 to 10,000 pg/kg/day; and the dosage for the Hydrangea extract is 1 to 1,000 mg/kg/day.

### Effects of Invention

As described above, the composition containing hydrangenol or a Hydrangea extract according to the present invention can increase the expression and activity of myoD, myogenin and mTOR and induce the formation of muscle fibers to increase muscle mass. It can also promote the gene expression and protein activity of muscle-related factors, including PPARδ and AMPK, and increase the ATP content in the cells to prevent muscle diseases, improve muscle functions, and enhance motor performance. Accordingly, the composition of the present invention can be used beneficially as a medical, food, or cosmetic composition.

### BRIEF DESCRIPTION OF DRAWINGS

FIGS. 1a, 1b and 1c show the results of HPLC analyses for hydrangenol contained in Hydrangea: FIG. 1a is the HPLC spectrum of a standard substance, hydrangenol, FIG. 1b is the HPLC spectrum of Hydrangea serrata, and FIG. 1c is the HPLC spectrum of Hydrangea macrophylla.

FIG. 2 is an ESIMS (positive-ion mode) spectrum of hydrangenol.

FIG. 3 is a ¹H-NMR spectrum of hydrangenol.

FIG. 4 is a ¹³C-NMR spectrum of hydrangenol.

FIG. 5 is a DEPT NMR spectrum of hydrangenol.

FIG. 6 is an HSQC NMR spectrum of hydrangenol.

FIG. 7 is an HMBC NMR spectrum of hydrangenol.

FIGS. 8a and 8b are a graph showing the muscle-related gene expression varied upon treatment with hydrangenol or a Hydrangea extract.

FIG. 9 is an image showing the muscle-related protein expression varied upon treatment with hydrangenol or a Hydrangea extract.

FIG. 10 is a graph showing the change in ATP concentration in a cell upon treatment with hydrangenol or a Hydrangea extract.

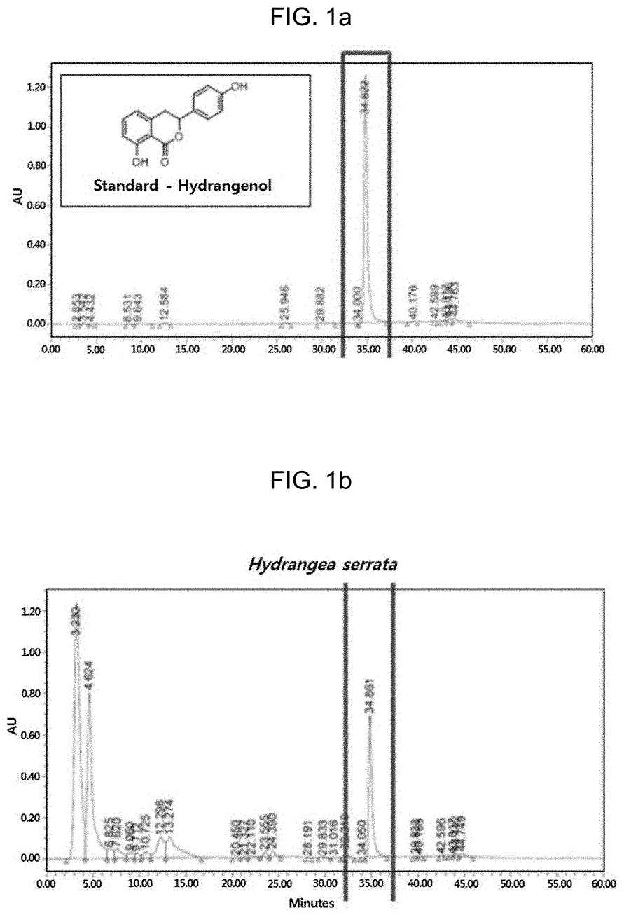

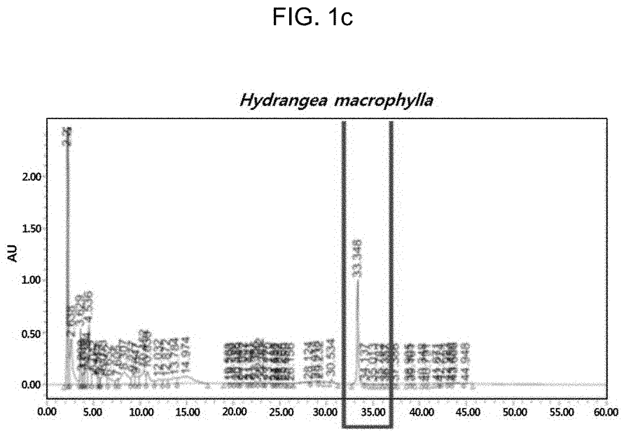

### BEST MODES FOR CARRYING OUT THE INVENTION

Hereinafter, the present invention will be described in further detail with reference to examples. It will be obvious to those skilled in the art that these examples are illustrative purposes only and are not construed to limit the scope of the present invention.

### EXAMPLE 1

### Preparation of Hydrangea Extract

The extract of Hydrangea in the composition of the present invention was prepared in the following procedures. Firstly, 20 kg of dried raw materials of Hydrangea serrata or Hydrangea macrophylla and 300 kg of purified water were mixed in an extraction tank and subjected to reflux extraction at 100° C. for 5 hours. The extracted sample was passed through a cartridge filter (10 μm), concentrated under reduced pressure and then spray-dried to yield a water-soluble powder.

### EXAMPLE 2

### Preparation of Hydrangenol Derived from Hydrangea Extract

The extract powder obtained in Example 1 was subjected to a gel filtration with a Diaion HP-20. Each 2 L of the mixed solutions of methanol (30%, 50%, 70%, 100%) and CH₂Cl₂-MeOH (1:1, v/v) was used as a developing solvent for solvent fractionation into five subfractions (392-70EDia 1˜5). The subfraction 392-70EDia4 (357.4 mg) was solvent-fractionized with Sephadex LH-20 and a developing solvent of methanol into seven subfractions (392-70EDia4a˜4g). Out of the subfractions, 392-70EDia4d was recrystallized in methanol to yield a pure amorphous compound 1 (hydrangenol).

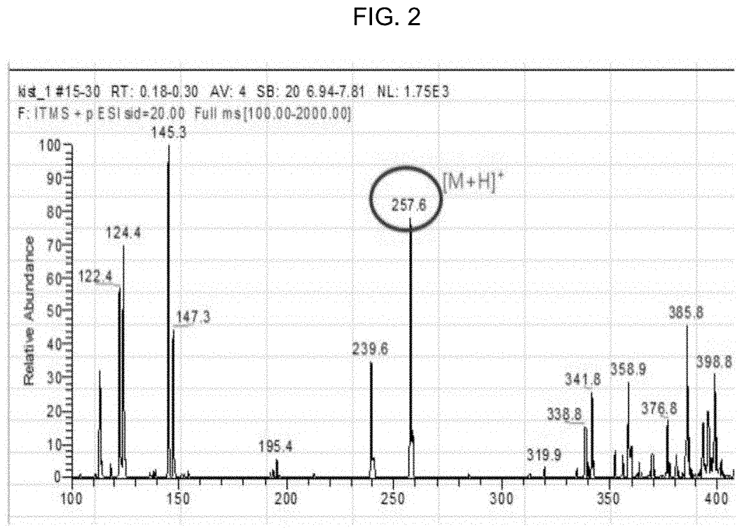

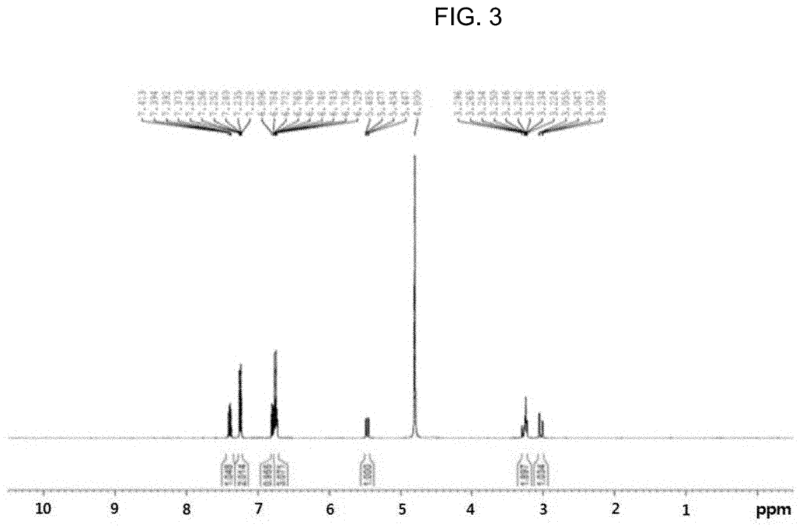

An ESIMS (positive-ion mode) analysis was firstly conducted to identify the structure of the product obtained in Example 2, suggesting that that m/z=257[M+H]⁺ (Refer to FIG. 2). As can be seen from the ¹H-NMR spectrum (Refer to FIG. 3), the methane proton (H-3) at δH 5.50 and the methylene proton (H-4) at δH 3.30 and 3.06 in strong magnetic field formed a vicinal coupling, and the chemical shift value as well as the vicinal coupling accounted for the protons being originated from the C-ring. As for the protons originated from the p-substituted benzene ring of a B-ring, the peaks H-2′ and H-3′ and the peaks H-6′ and H-5′ formed ortho couplings and showed up as a doublet (J=8.4 Hz); and the peaks H-2′ and H-6′ and the peaks H-3′ and H-5′ also formed ortho couplings and showed up as a doublet. This implicitly indicated that the product had a structure symmetric with respect to the hydroxyl group. In the 1,2,3-trisubstituted benzene of the A-ring, the protons H-5 and H-7 independently formed a coupling with the proton H-6. Hence, the protons H-5 and H-7 appeared as a doublet with the ortho couplings, and the proton H-6 showed up as a double of doublets with the meta couplings, suggesting that all the peaks corresponded to one proton.

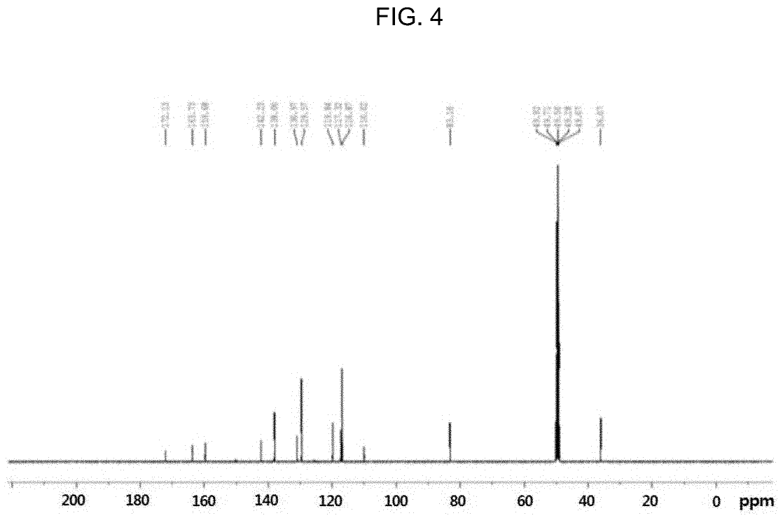

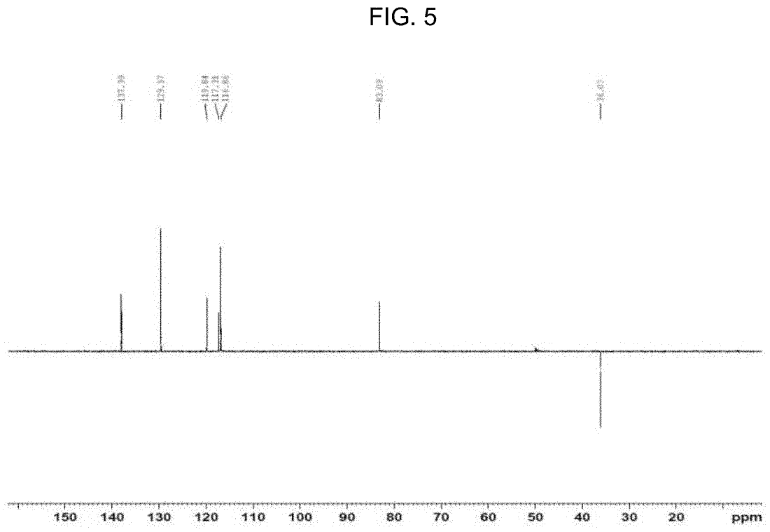

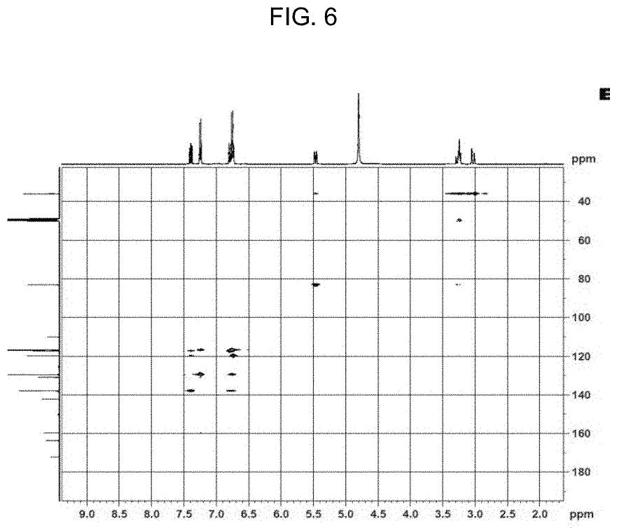

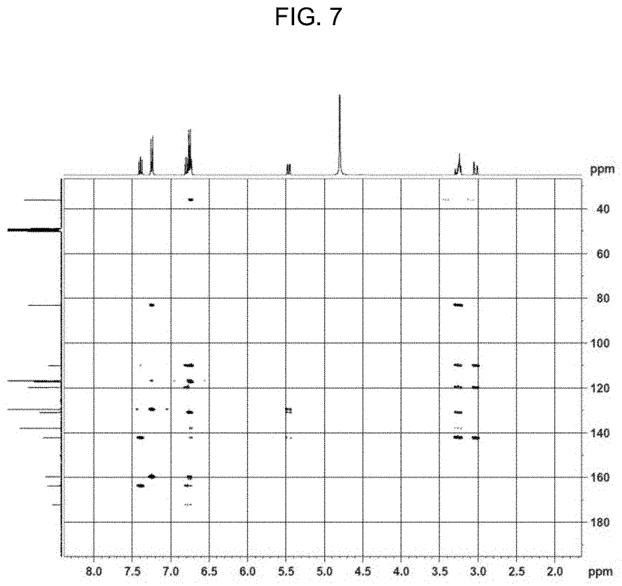

In the ¹³C-NMR spectrum (Refer to FIG. 4), fifteen peaks including a para-substituent appeared. It was interpreted as follows: the quaternary carbon peak at δC 172 was originated from the first carbon of the compound 1, that is, on the carbonyl group; and the peaks at δC 36.1 and δC 83.1 were originated from an aliphatic carbon and an oxygenated carbon, respectively. In addition, the DEPT NMR spectrum (Refer to FIG. 5) identified seven protonated carbons, showing that the peak at δC 36.1 was a methylene group originated from the C-4. A 2D NMR analysis was carried out to analyze the precise structures of the peaks. The precise positions of the peaks were addressed according to the HSQC (Refer to FIG. 6), and the bonding positions of substituents were determined from the HMBC (Refer to FIG. 7). That is, the peak at δH 7.26 (2H, d, J=8.4 Hz, H-2′,6′) had a correlation with the peak of C-4 at δC 36.1; whereas the peaks at δH 3.06 and δH 3.30 originated from H-4 had a correlation with the peaks at δC 83.1 (C-3), δC 119.8 (C-5), δC 110.0 (C-9), and δC 142.2 (C-10). A summary of the results and a comparison with the literatures identified the compound of Example 2 as hydrangenol (Yoshikawa M., Matsuda H., Shimoda H., Shimada H., Harada E., Naitoh Y., Miki A., Yamahara J., Murakami N. Development of Bioactive Functions in Hydrangeae Dulcis Folium. V. On the Antiallergic and Antimicrobial Principles of Hydrangeae Dulcis Folium. (2). Thunberginols C, D, and E, Thunberginol G 3′-O-Glucoside, (−)-Hydrangenol 4′-O-Glucoside, and (+)-Hydrangenol 4′-O-Glucoside. Chem. Pharm. Bull. 1996, 44: 1440-1447).

### EXPERIMENTAL EXAMPLE 1

### Change in Muscle-Related Gene Expression Upon Treatment with Hydrangenol or Hydrangea Extract

Each sample obtained in Examples 1 and 2 was analyzed in regards to its effects on the change in gene expression involved in the gain of muscle mass and the mitochondrial biogenesis and electron transport chain (ETC). For this experiment, C2C12 cells were differentiated for 5 days and then treated with the sample of Example 1 (25 μg/ml) or Example 2 (2.5 ug/ml) for 1 hour or 24 hours, respectively. Then, Trizol was used to extract RNA from the cell, and cDNA was synthesized from the RNA (cDNA synthesis kit, Bio-Rad). Finally, a realtime PCR (qRT-PCR) was performed (Viia7, Agilent Biosystems) using the oligomers of Table 1 as primers.

TABLE 1

| | | | SEQ |
|---|---|---|---|
| Gene | Direction | Sequence (5′→3′) | ID NO |
| PGC-1a | Forward | GTCCTTCCTCCATGCCTGAC | 1 |
| | Reverse | GACTGCGGTTGTGTATGGGA | 2 |
| ERRa | Forward | GAGGTGGACCCTTTGCCTTT | 3 |
| | Reverse | GGCTAACACCCTATGCTGGG | 4 |
| NRF-1 | Forward | CTTCATGGAGGAGCACGGAG | 5 |
| | Reverse | ATGAGGCCGTTTCCGTTTCT | 6 |
| Tfam | Forward | GAGCGTGCTAAAAGCACTGG | 7 |
| | Reverse | CCACAGGGCTGCAATTTTCC | 8 |
| MyoD | Forward | CCGTGTTTCGACTCACCAGA | 9 |
| | Reverse | GTAGTAGGCGGTGTCGTAGC | 10 |
| Myogenin | Forward | GAGGAAGTCTGTGTCGGTGG | 11 |
| | Reverse | CCACGATGGACGTAAGGGAG | 12 |
| SDHb | Forward | ACTGGTGGAACGGAGACAAG | 13 |
| | Reverse | GTTAAGCCAATGCTCGCTTC | 14 |
| CytC | Forward | GGGCCTCGTTAGTGCAGCAGG | 15 |
| | Reverse | GGGCTCCCAGAAAAGGTTGCCT | 16 |
| COX2 | Forward | ACGAAATCAACAACCCCGTA | 17 |
| | Reverse | GGCACAACGACTCGGTTATC | 18 |
| COX4 | Forward | ACTACCCCTTGCCTGATGTG | 19 |
| | Reverse | GCCCACAACTGTCTTCCATT | 20 |
| ATP5g1 | Forward | TGGGGACCAGGGCAGCCATT | 21 |
| | Reverse | AGGGCTTGCTGCCCACACAT | 22 |
| PPAra | Forward | ATCCAGGGTTCAGTCCAGTG | 23 |
| | Reverse | GCTTAGGGACAGTGACAGGT | 24 |
| PPARd | Forward | GTTCCTCTGACCCTGTCCTC | 25 |
| | Reverse | CCAGCAAGTTTCAAGCCACT | 26 |
| UCP1 | Forward | CGGAAACAAGATCTCAGCCG | 27 |
| | Reverse | CTGACCTTCACGACCTCTGT | 28 |
| SIRT1 | Forward | TGCCATCATGAAGCCAGAGA | 29 |
| | Reverse | AACATCGCAGTCTCCAAGGA | 30 |
| GAPDH | Forward | AACTTTGGCATTGTGGAAGG | 31 |
| | Reverse | GGATGCAGGGATGATGTTCT | 32 |

In the experimental example, the extract of Hydrangea serrata is indicated as S; the extract of Hydrangea macrophylla as M; and hydrangenol as H. “0” means a control that was treated with vehicle.

The samples of Examples 1 and 2 increase the amount of mRNA expression of peroxisome proliferator-activated receptor a (PPARα) in the skeletal muscle and promote peroxisomal β-oxidation along with PGC-1α for use as an energy source in the muscle, thereby enhancing muscle functions. In addition, the samples of Examples 1 and 2 activate PGC-1α and peroxisome proliferator-activated receptor 5 (PPARδ) along with sirtuin 1 (SIRT 1) of which expression is increased, thus enhancing absorption of glucose, oxidation of fatty acids, and mitochondrial synthesis.

MyoD is a crucial protein involved in muscle differentiation, which means that the hydrangenol of Hydrangea can accelerate the differentiation of muscle cells. Myogenin is a protein involved in skeletal muscle development as a muscle-specific transcription factor. An increase in the mRNA expression of the myogenic regulatory factor protein implies inducing the differentiation of muscle cells and the synthesis of proteins and increasing the muscle mass.

Nuclear respiratory factor 1 (NRF-1) is a transcription factor that activates the expression of proteins involved in cell growth and cellular respiration. An increase in the expression of NRF-1 results in the expression of genes regulating cellular growth, respiration, and mitochondrial DNA (mtDNA) transcription and replication. The NRF-1 is known to regulate mitochondrial transcription factor A (TFAM), which is a transcription factor of mitochondria that participates in mitochondrial genome replication and transcription. In addition, an increase in mitochondrial cytochrome C (CytC), cytochrome C oxidase subunit 2 (Cox2), and cytochrome C oxidase subunit 4 (Cox4) indicates the activation of the mitochondrial electron transport chain (ETC). A mitochondrial ATP synthase lipid-binding protein (ATG5g1) is a part of the ATP synthase of mitochondria. An increase in the gene expression of ATG5g1 results in increasing the intracellular content of ATP acting as an energy carrier in cells, which eventually boosts the activity of the cells.

As factors involved in muscle hypertrophy, tuberous sclerosis complex 1 (TSC1) and tuberous sclerosis complex 2 (TSC2) are GTPase-activating proteins (GAPs) that negatively regulate the mammalian target of rapamycin (mTOR). The mammalian target of rapamycin (mTOR) is a phosphorylated protein that controls cell growth and proliferation, cell survival rate, and protein synthesis and transcription. A decrease in the mRNA expression of TSC1 and TSC2 is activating the mTOR signaling, which increases the protein synthesis in cells and promotes the cell growth and the activity of cells, increasing the muscle cells.

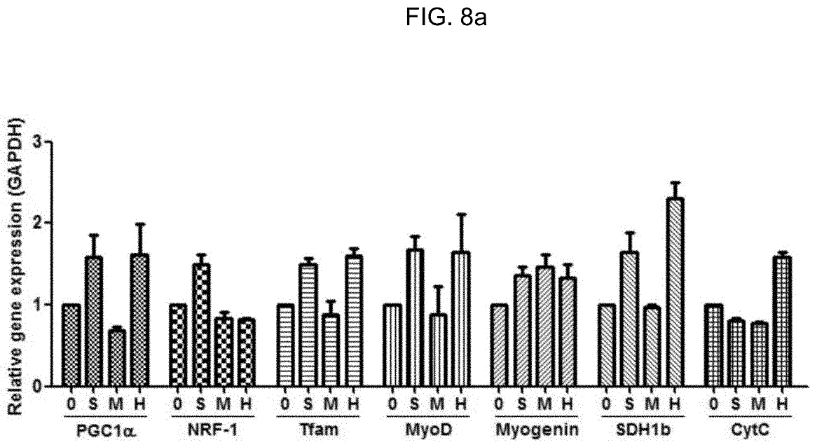

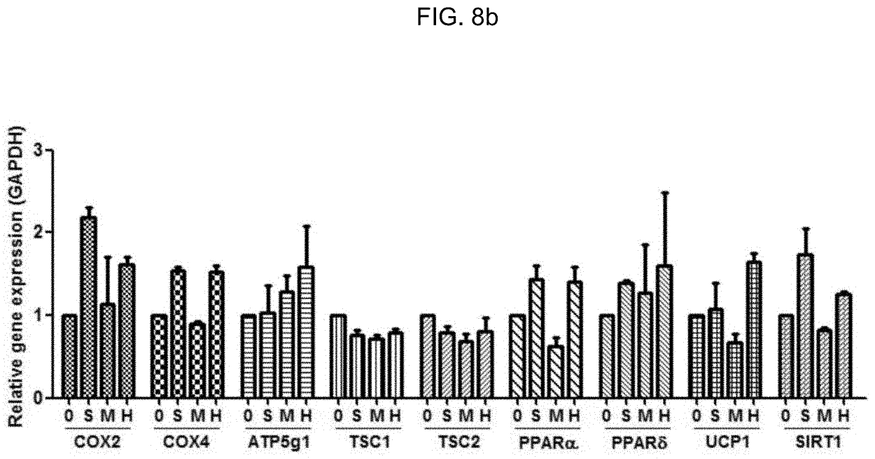

It was therefore confirmed, as shown in FIG. 8, that especially, Hydrangea serrata extract S in Example 1 induced the gene expression of PGC1α, NRF-1, TFAM, MyoD, PPARα, PPARδ, ATG5g1, and SIRT1. It suggested that the absorption of glucose and fatty acids and the mitochondrial protein synthesis increased the ATP content and helped to enhance the motor performance. That was also backed up by the increased expression of SDH1b, CyctC, COX2, COX4, ATP5g1, UCP1, NRF-1, and PPARδ and the decreased expression of TSC1 and TSC2. In addition, Hydrangea macrophylla extract M increased the expression of Myogenin, COX2, ATP5g1, and PPARδ and helped to gain the muscle mass and enhance muscle functions through boosted muscle development and activation of mitochondrial electron transport chain (ETC), or the like.

The sample of Example 2, which was the effective ingredient of Example 1, regulated the amount of expression for most of genes shown in FIG. 8 and particularly promoted the expression of MyoD and Myogenin to affect the cell proliferation and the protein synthesis, thereby inducing a gain of the muscle mass.

### EXPERIMENTAL EXAMPLE 2

### Change in Muscle-Related Protein Expression Upon Treatment with Hydrangenol or Hydrangea Extract

Each sample obtained in Examples 1 and 2 was analyzed in regards to its effects on the change in protein expression involved in the prevention of muscle diseases, the improvement of muscle functions, and the promotion of motor performance. For this experiment, C2C12 cells were differentiated for 5 days and then treated with the sample of Example 1 (25 ug/ml) or Example 2 (2.5 ug/ml) for 1 hour or 24 hours, respectively. Then, the cell was broken down with a modified LIPA buffer, and 20 ug of each sample was used for analysis. The primary antibodies of p-ERK (9101S: Cell Signaling), p-mTOR (ab109268, Abcam), mTOR (2972, Cell Signaling), p-p70S6K (9234, Cell Signaling), p-AMPK (4186, Cell Signaling), PGC1α (ab54481, Abcam), and β-actin (A5316, Sigma) were used for the analysis.

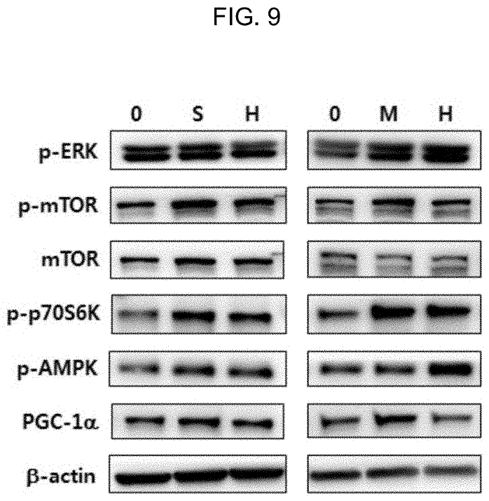

As can be seen from FIG. 9, the samples obtained in Examples 1 and 2 increased the activity of p-AMPK and the protein expression of PGC1α. Further, in the same manner as indicated by the results of the Experimental Example 1, the absorption of glucose and fatty acids and the mitochondrial synthesis increased the ATP content and helped to enhance the motor performance. In addition, the samples of Examples 1 and activated the phosphorylation of mTOR through the phosphorylation of ERK, also known as MAPK, and the mTOR signaling followed by the phosphorylation of the subordinate protein, p70S6K, to affect the proliferation of muscle cells and the protein synthesis, inducing an increase in the muscle mass.

### EXPERIMENTAL EXAMPLE 3

### Change in Intracellular ATP Concentration Upon Treatment with Hydrangenol or Hydrangea Extract

The sample obtained in Example 1 was analyzed in regards to its effect on the change in intracellular ATP concentration. A mitochondrial ToxGlo™ Assay (Promega) kit was used to measure the amount of ATP in the cells treated with the sample of Example 1. The differentiated cells were treated with a 2X ATP detection reagent at the room temperature and measured in regards to the luminescence.

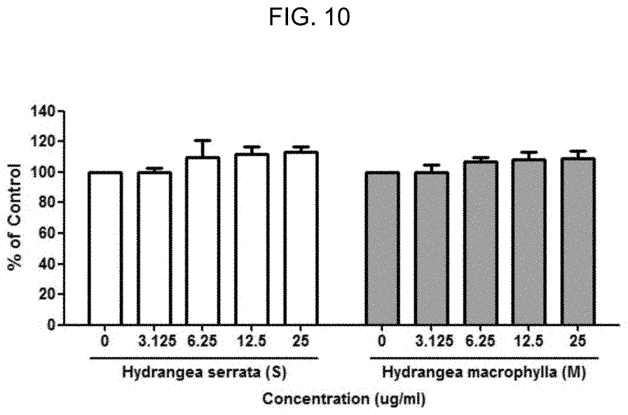

As can be seen from FIG. 10, the sample of Example 1 increased the amount of ATP in the cells in a concentration-dependent manner. Therefore, in the same manner as described in Experimental Examples 1 and 2, the sample of Example 1 increased the activity of mitochondria and helped to enhance the motor performance.

### FORMULATION EXAMPLE 1

### Preparation of Tablets

The extract of Example 1 or Hydrangenol of Example 2 was mixed with the ingredients of Table 2 or 3 and processed into tablets according to a general preparation method for tablet.

TABLE 2

| Ingredients | Unit weight (mg) |
|---|---|
| Example 1 | 50 |
| Corn starch | 100 |
| Lactose | 100 |
| Stearic acid | 2 |

TABLE 3

| Ingredients | Unit weight (mg) |
|---|---|
| Example 2 | 10 |
| Corn starch | 100 |
| Lactose | 100 |
| Stearic acid | 2 |

### FORMULATION EXAMPLE 2

### Preparation of Capsules

The extract of Example 1 or Hydrangenol of Example 2 was mixed with the ingredients of Table 4 or 5 and filled in gelatin capsules to prepare soft capsules according to a general preparation method for capsule.

TABLE 4

| Ingredients | Unit weight (mg) |
|---|---|
| Example 1 | 50 |
| Corn starch | 100 |
| Lactose | 100 |
| Stearic acid | 2 |

TABLE 5

| Ingredients | Unit weight (mg) |
|---|---|
| Example 2 | 2 |
| Vitamin E | 2.25 |
| Vitamin C | 2.25 |
| Palm oil | 0.5 |
| Vegetable hydrogenated oil | 2 |
| Yellow lead | 1 |
| Lecithin | 2.25 |
| Filling solution for soft capsule | 387.75 |

### FORMULATION EXAMPLE 3

### Preparation of Liquid

The extract of Example 1 or Hydrangenol of Example 2 was mixed with the ingredients of Table 6 or 7 and filled in a bottle or a pouch to prepare a liquid according to a general preparation method for beverage.

TABLE 6

| Ingredients | Unit weight (g) |
|---|---|
| Example 1 | 2.5050 |
| Xanthan gum | 0.0075 |
| Pructooligosaccharide | 0.7500 |
| Powdered coconut flower nectar | 1.0500 |
| Concentrated ssangwha-tang | 1.5000 |
| Red ginseng flavor | 0.0450 |
| Purified water | 9.1425 |

TABLE 7

| Ingredients | Unit weight (g) |
|---|---|
| Example 2 | 0.0205 |
| Xanthan gum | 0.0075 |
| Pructooligosaccharide | 0.7500 |
| Powdered coconut flower nectar | 1.0500 |
| Concentrated ssangwha-tang | 1.5000 |
| Red ginseng flavor | 0.0450 |
| Purified water | 20.1425 |

### FORMULATION EXAMPLE 4

### Preparation of Chewable Gel

The extract of Example 1 or Hydrangenol of Example 2 was mixed with the ingredients of Table 8 or 9 and filled in a three-sided seal pouch to prepare a chewable gel according to a general preparation method for chewable gel.

TABLE 8

| Ingredients | Unit weight (g) |
|---|---|
| Example 1 | 2.0000 |
| Food gel | 0.3600 |
| Carrageenan | 0.0600 |
| Calcium lactate | 0.1000 |
| Sodium citrate | 0.0600 |
| Complex scutellaria extract | 0.0200 |
| Enzymatically modified stevia | 0.0440 |
| Fructooligosaccharide | 5.0000 |
| Red grape concentrate | 2.4000 |
| Purified water | 13.9560 |

TABLE 9

| Ingredients | Unit weight (g) |
|---|---|
| Example 2 | 0.0200 |
| Food gel | 0.3600 |
| Carrageenan | 0.0600 |
| Calcium lactate | 0.1000 |
| Sodium citrate | 0.0600 |
| Complex scutellaria extract | 0.0200 |
| Enzymatically modified stevia | 0.0440 |
| Fructooligosaccharide | 5.0000 |
| Red grape concentrate | 2.4000 |
| Purified water | 13.9560 |

### FORMULATION EXAMPLE 5

### Preparation of Nutrient Cream

The extract of Example 1 or Hydrangenol of Example 2 was processed into the composition of Table 10 or 11 according to a general preparation method for nutrient cream.

TABLE 10

| Ingredients | Content (%) |
|---|---|
| Example 1 | 1.0 |
| Sitosterol | 4.0 |
| Polyglyceryl 2-oleate 3.0 | 3.0 |
| Ceteareth-4 | 2.0 |
| Cholesterol | 3.0 |
| Dicetyl phosphate | 0.4 |
| Concentrated glycerin | 5.0 |
| Sunflower oil | 22.0 |
| Carboxylvinyl polymer | 0.5 |
| Triethanol amine | 0.5 |
| Preservative | trace |
| Flavor | trace |
| Purified water | balance |

TABLE 11

| Ingredients | Content (%) |
|---|---|
| Example 2 | 0.01 |
| Sitosterol | 4.0 |
| Polyglyceryl 2-oleate 3.0 | 3.0 |
| Ceteareth-4 | 2.0 |
| Cholesterol | 3.0 |
| Dicetyl phosphate | 0.4 |
| Concentrated glycerin | 5.0 |
| Sunflower oil | 22.0 |
| Carboxylvinyl polymer | 0.5 |
| Triethanol amine | 0.5 |
| Preservative | trace |
| Flavor | trace |
| Purified water | balance |

The above-defined composition is given as a formulation example using a mixture of appropriate compositions. Yet the mixing ratio and the ingredients maybe varied arbitrarily under necessity.

The extract of the present invention was stable under the testing conditions for all formulation examples and hence not problematic in the stability of the dosage form.

1. A composition for preventing and treating a muscle disease, improving a muscle function, or enhancing a motor performance, the composition comprising hydrangenol of the following Chemical Formula 1 or a Hydrangea extract containing the hydrangenol as an active ingredient:

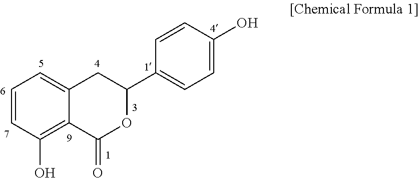 [SMILES: O=C1OC(c2ccc(O)cc2)Cc2cccc(O)c21]

2. The composition according to claim 1, wherein the hydrangenol is isolated from the Hydrangea extract.

3. The composition according to claim 1, wherein the Hydrangea extract is obtained by extraction with at least one solvent selected from the group consisting of water, an organic solvent, or a combination of both.

4. The composition according to claim 1, wherein the hydrangenol or the Hydrangea extract is contained in an amount of 0.0001 to 100 wt. % with respect to the total weight of the composition.

5. The composition according to claim 1, wherein the composition has at least one dosage form selected from the group consisting of tablet, granule, pill, capsule, liquid, jelly, and gum, wherein the composition is for oral application.

6. The composition according to claim 1, wherein the composition has at least one dosage form selected from the group consisting of toner, essence, nourishing cream, moisturizing cream, spot, gel, lotion, ointment, patch, and aerosol, wherein the composition is for cutaneous application.

7. The composition according to claim 1, wherein the composition is a medical composition, a food composition, a quasi-drug composition, or a cosmetic composition.

8. The composition according to claim 1, wherein the muscle disease is at least one disease selected from the group consisting of muscular atrophy, muscular dystrophy, myasthenia, muscle degeneration, and sarcopenia.
